(19) United States
(12) Patent Application Publication
(10) Pub. No.: US 2024/0358325 A1
(43) Pub. Date: Oct. 31, 2024

(54) BLOOD VESSEL LOCATOR

(71) Applicant: Ohio State Innovation Foundation, Columbus, OH (US)

(72) Inventors: Sandra Metzler, Columbus, OH (US); Hannah Richards, Columbus, OH (US); Siddharth Fernandes, Columbus, OH (US); Mackenzie Crawford, Columbus, OH (US); Aarti Patel, Columbus, OH (US); Rene Hardin, Columbus, OH (US); Dylan Trainor, Columbus, OH (US); Alejandro Bresolin, Columbus, OH (US)

(21) Appl. No.: 18/646,287

(22) Filed: Apr. 25, 2024

Related U.S. Application Data
(60) Provisional application No. 63/498,150, filed on Apr. 25, 2023.

(51) Int. Cl. A61B 5/00

(52) U.S. Cl. CPC A61B 5/489; A61B 5/0082; A61B 2503/045; A61B 2560/0443; A61B 2562/0233

(57) ABSTRACT

A blood vessel location device may include a housing. A blood vessel location device may include a light emitting diode (LED) strip coupled to the housing and including a linear array of LEDs, the LED strip moveable between a use position and a storage position relative to the housing. A blood vessel location device may include a control system positioned within the housing and configured to control the LEDs.

### CROSS-REFERENCE TO RELATED APPLICATIONS

This application claims the benefit of U.S. Provisional Patent Application No. 63/498,150 filed on Apr. 25, 2023, the entire contents of which are incorporated herein by reference.

### BACKGROUND

This disclosure relates to blood vessel locators. More particularly, this disclosure relates to blood vessel locators and system for infants and neonates.

### SUMMARY

In some aspects, the techniques described herein relate to a blood vessel location device, including: a housing; a light emitting diode (LED) strip coupled to the housing and including a linear array of LEDs, the LED strip moveable between a use position and a storage position relative to the housing; and a control system positioned within the housing and configured to control the LEDs.

In some aspects, the techniques described herein relate to a blood vessel location device, further including pegs coupling the LED strip to the housing, the pegs spacing the LED strip away from the housing in the use position.

In some aspects, the techniques described herein relate to a blood vessel location device, further including two pegs hingedly coupled between the LED strip and the housing.

In some aspects, the techniques described herein relate to a blood vessel location device, wherein the LED strip includes at least two apertures, the blood vessel location system further including: two pegs, each including a stud sized to be received within one of the at least two apertures of the LED strip; and two hinges coupling the pegs to the housing.

In some aspects, the techniques described herein relate to a blood vessel location device, wherein the studs are interference fit with the apertures.

In some aspects, the techniques described herein relate to a blood vessel location device, further including a cable connecting the LED strip to the control system.

In some aspects, the techniques described herein relate to a blood vessel location device, wherein the LED strip is removable from the housing.

In some aspects, the techniques described herein relate to a blood vessel location device, wherein the linear array of LEDs includes white LEDs.

In some aspects, the techniques described herein relate to a blood vessel location device, wherein the linear array of LEDs includes red LEDs.

In some aspects, the techniques described herein relate to a blood vessel location device, wherein the linear array of LEDs includes white LEDs in a linear arrangement and red LEDs in a linear arrangement.

In some aspects, the techniques described herein relate to a blood vessel location device, wherein the control system includes a LED switch configured to select the white LEDs or the red LEDs.

In some aspects, the techniques described herein relate to a blood vessel location device, wherein the linear array of LEDs includes a plurality of white LEDs in a linear arrangement and a plurality of red LEDs in a linear arrangement.

In some aspects, the techniques described herein relate to a blood vessel location device, wherein the linear array of LEDs includes equidistantly spaced LEDs.

In some aspects, the techniques described herein relate to a blood vessel location device, wherein the LED strip is formed of a translucent plastic.

In some aspects, the techniques described herein relate to a blood vessel location device, wherein the LED strip is formed of a flexible plastic.

In some aspects, the techniques described herein relate to a blood vessel location device, including: a housing; a light emitting diode (LED) strip formed of a flexible plastic, the LED strip coupled to the housing and including a linear array of white LEDs and a linear array of red LEDs, a set of inner apertures, and a set of outer apertures; two pegs selectively coupled with the inner apertures or the outer apertures, the pegs spacing the LED strip away from the housing, wherein the LED strip provides a first compliance when the pegs are engaged with the inner apertures and a second compliance when the pegs are engaged with the outer apertures; and a control system positioned within the housing and including a LED switch configured to select the white LEDs or the red LEDs.

In some aspects, the techniques described herein relate to a blood vessel location device, wherein the LED strip is connected to the control system via a cable and moveable between a use position and a storage position relative to the housing.

In some aspects, the techniques described herein relate to a method of locating a blood vessel of an infant, the method including: supporting a light emitting diode (LED) strip with a housing; illuminating a linear array of LEDs through a body part of the infant; and moving the LED strip between a use position and a storage position relative to the housing.

In some aspects, the techniques described herein relate to a method, further including illuminating either a linear array of white LEDs or a linear array of red LEDs through the body part of the infant.

In some aspects, the techniques described herein relate to a method, wherein moving the LED strip between the use position and the storage position includes actuating hinges and pegs coupled between the housing and the LED strip. This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a perspective view of a blood vessel locator, according to some embodiments.

FIG. 2 is a perspective view of a peg of the blood vessel locator of FIG. 1, according to some embodiments.

FIG. 3 is a top view of the peg of FIG. 2, according to some embodiments.

FIG. 4 is a front view of the peg of FIG. 2, according to some embodiments.

FIG. 5 is a side view of the peg of FIG. 2, according to some embodiments.

FIG. 6 is a perspective view of a LED strip of the blood vessel locator of FIG. 1, according to some embodiments.

FIG. 7 is a top view of the LED strip of FIG. 6, according to some embodiments.

FIG. 8 is a section view of the LED strip taken along line A-A of FIG. 7.

### DETAILED DESCRIPTION

Following below are more detailed descriptions of concepts related to, and implementations of, methods, apparatuses, and systems for a blood vessel locator. Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring to the figures generally, the various embodiments disclosed herein relate to systems, apparatuses, and methods for a blood vessel locator. Embodiments discussed below are particularly useful for infant blood vessel location and more particularly for neonates. Neonates are children born at the gestational age of 37 weeks or less. Infants, and especially neonates, are susceptible to complications including respiratory distress syndrome, infection, and traumatic birth injury due to the lack of a fully developed immune system and endochondral ossification. Infants diagnosed with these and similar conditions should readily receive adequate and intensive treatment. Neonate blood vessels are small, fragile, and more susceptible to rupturing when during vascular access procedures (VAPs) than adults. Repeated failed attempts generate considerable medical waste in addition to both causing discomfort for the infant and consuming time, which may delay necessary treatment for the infant. A general inability to visualize the infant's blood vessels complicates treatment.

Vascular visibility is advantageous for precise vascular access procedures including the direct method of introducing or extracting devices or chemicals into the blood vessels of the infant. Infants have more delicate vessels than adults, and also have varying physiology. While fully developed infants may have a substantial dermal fat layer, neonates (for example) can have more delicate skin that has only started to keratinize. While commercially available vein finder products exist, they are not cost-effective or durable and obstruct the nurses' access procedure. Commercially available vein finders include light emitting diodes (LEDs) that inadequately penetrate through the infants' subcutaneous fat layer leading nurses to guess the location of vessels, defeating the purpose of the device. Durability of construction is often lacking leading to frequent breakage, the contour and/or geometry of commercially available products can be cumbersome (e.g., shape causes the infant's arm to slip off of the device, or promotes unstable VAP needle placement). Some devices include LEDs that can temporarily blind nurses if the infant's hand slips off LEDs.

Therefore, there exists a need for a device that can effectively increase the visibility of arteries and veins in premature full-term babies, while also decreasing the amount of time spent in these procedures.

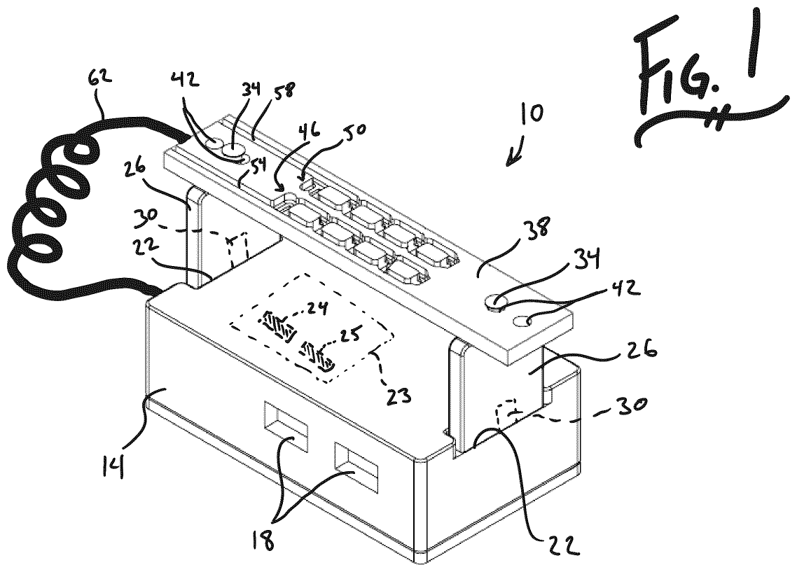

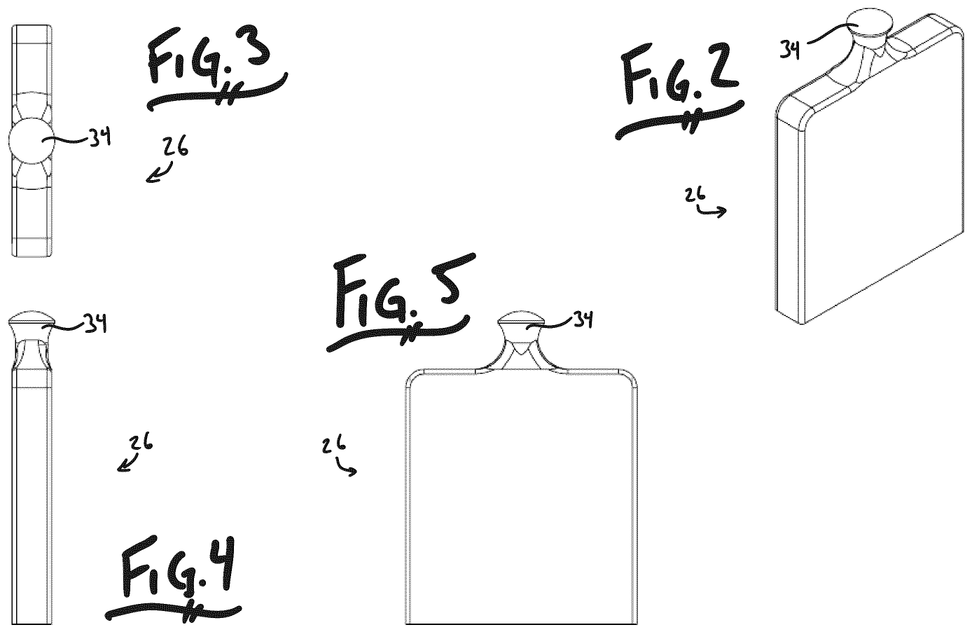

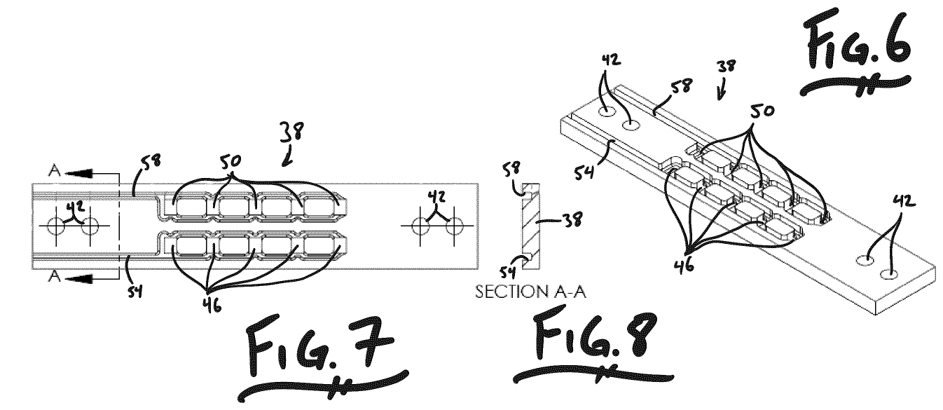

As shown in FIGS. 1-8, a blood vessel location system 10 includes a base housing 14 defining two switch apertures 18 and two hinge rabbits 22. The base housing 14 is sized to contain a control system 23 including a power system 24 (e.g., batteries, a mains power connection, etc.) and an LED control circuit 25. The power system 24 includes a power switch positioned in one of the switch apertures 18 and the LED control circuit 25 includes a LED switch positioned in the other of the switch apertures 18. In some embodiments, the base housing 14 is formed from a plastic material.

Two pegs 26 are coupled to the hinge rabbits 22 of the base housing 14 by hinges 30. The pegs 26 are sized to be received within the corresponding hinge rabbit 22 of the base housing 14 and are movable between a use position (shown in FIG. 1) and a storage position where the hinges 30 are actuated to lower the pegs 26 so they are partially recessed into the hinge rabbits 22. The pegs 26 each include studs 34 that extend generally upward. In some embodiments, the studs 34 define a button shape with a relatively wide head and a relatively narrow neck region. In some embodiments, the pegs 26 are formed from a plastic material.

An LED strip 38 is supported by the pegs 26 and defines four mounting apertures 42 sized to receive the studs 34 via interference fit. In some embodiments, the four apertures 42 may be utilized to adjust the compliance of the LED strip 38. For example, the widest apertures 42 may be used to provide a more compliant LED strip 38 and the narrowest apertures may be used to provide a less compliant LED strip 38. In some embodiments the LED strip 38 is formed from a translucent plastic.

Five red LEDS 46 are provided on a first side of the LED strip 38. In some embodiments, the five red LEDS 46 are provided in an equally spaced linear array. In some embodiments, the red LEDS 46 are positioned with unequal spacing or in a bank of two LEDS. In some embodiments, more than five or less than five red LEDS 46 are included.

Five white LEDS 50 are provided on a second side of the LED strip 38. In some embodiments, the five white LEDS 50 are provided in an equally spaced linear array. In some embodiments, the white LEDS 50 are positioned with unequal spacing or in a bank of two LEDS. In some embodiments, more than five or less than five white LEDS 50 are included.

A first wire recess 54 and a second wire recess 58 are formed in the LED strip 38 to provide a space for power and control wires to the LED arrays 46, 50. A cable 62 connects the control system 23 positioned in the base housing 14 and the LED strip 38.

In operation, an operator engages the power switch of the power circuit 24 to power on the blood vessel locator 10 and selects either the red LEDs 46 or the white LEDS 50 using the LED switch of the LED circuit 25. A patient body part (e.g., a wrist, arm, hand, etc.) is then laid across the LED strip 38 and the selected LEDS 46, 50 shown therethrough to aid in blood vessel visualization. The user can arrange the blood vessel locator 10 in either the use position as shown in FIG. 1, or in the storage position with the hinges 30 lowered to provide a more compact storage arrangement. In some embodiments, the LED strip 38 can be removed from the pegs 26 and used without the base housing 14 (e.g., used in the hand of a nurse). In some embodiments, the user can adjust the intensity of the LEDs via the control system 23. In some embodiments, the LED strip 38 is touch sensitive such that the LEDs only illuminate when in contact with skin. In some embodiments, the LED strip 38 includes load sensors or pressure sensors that detect how hard a user is pressing on the LED strip 38 and increases illumination intensity as the pressure or load increases.

Additional details and advantages are discussed in detail in the Appendix attached hereto.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

What is claimed is:

1. A blood vessel location device, comprising:
a housing;
a light emitting diode (LED) strip coupled to the housing and including a linear array of LEDs, the LED strip moveable between a use position and a storage position relative to the housing; and
a control system positioned within the housing and configured to control the LEDs.

2. The blood vessel location device of claim 1, further comprising pegs coupling the LED strip to the housing, the pegs spacing the LED strip away from the housing in the use position.

3. The blood vessel location device of claim 1, further comprising two pegs hingedly coupled between the LED strip and the housing.

4. The blood vessel location device of claim 1, wherein the LED strip includes at least two apertures, the blood vessel location system further comprising:
two pegs, each including a stud sized to be received within one of the at least two apertures of the LED strip; and
two hinges coupling the pegs to the housing.

5. The blood vessel location device of claim 4, wherein the studs are interference fit with the apertures.

6. The blood vessel location device of claim 1, further comprising a cable connecting the LED strip to the control system.

7. The blood vessel location device of claim 1, wherein the LED strip is removable from the housing.

8. The blood vessel location device of claim 1, wherein the linear array of LEDs includes white LEDs.

9. The blood vessel location device of claim 1, wherein the linear array of LEDs includes red LEDs.

10. The blood vessel location device of claim 1, wherein the linear array of LEDs includes white LEDs in a linear arrangement and red LEDs in a linear arrangement.

11. The blood vessel location device of claim 10, wherein the control system includes a LED switch configured to select the white LEDs or the red LEDs.

12. The blood vessel location device of claim 1, wherein the linear array of LEDs includes a plurality of white LEDs in a linear arrangement and a plurality of red LEDs in a linear arrangement.

13. The blood vessel location device of claim 1, wherein the linear array of LEDs includes equidistantly spaced LEDs.

14. The blood vessel location device of claim 1, wherein the LED strip is formed of a translucent plastic.

15. The blood vessel location device of claim 1, wherein the LED strip is formed of a flexible plastic.

16. A blood vessel location device, comprising:
a housing;
a light emitting diode (LED) strip formed of a flexible plastic, the LED strip coupled to the housing and including a linear array of white LEDs and a linear array of red LEDs, a set of inner apertures, and a set of outer apertures;
two pegs selectively coupled with the inner apertures or the outer apertures, the pegs spacing the LED strip away from the housing, wherein the LED strip provides a first compliance when the pegs are engaged with the inner apertures and a second compliance when the pegs are engaged with the outer apertures; and
a control system positioned within the housing and including a LED switch configured to select the white LEDs or the red LEDs.

17. The blood vessel location device of claim 16, wherein the LED strip is connected to the control system via a cable and moveable between a use position and a storage position relative to the housing.

18. A method of locating a blood vessel of an infant, the method including:
supporting a light emitting diode (LED) strip with a housing;
illuminating a linear array of LEDs through a body part of the infant; and
moving the LED strip between a use position and a storage position relative to the housing.

19. The method of claim 18, further comprising illuminating either a linear array of white LEDs or a linear array of red LEDs through the body part of the infant.

20. The method of claim 18, wherein moving the LED strip between the use position and the storage position includes actuating hinges and pegs coupled between the housing and the LED strip.
